(19) United States
(12) Patent Application Publication
(10) Pub. No.: US 2025/0194914 A1
(43) Pub. Date: Jun. 19, 2025

(54) SYSTEMS AND METHODS FOR ORAL INSPECTION

(71) Applicant: Dyson Technology Limited, Malmesbury, Wiltshire (GB)

(72) Inventors: Samuel Ian Harold ACKROYD, Bristol (GB); Matthew Jonathan STUBBS, Newport (GB)

(21) Appl. No.: 18/844,497

(22) PCT Filed: Mar. 2, 2023
(86) PCT No.: PCT/GB2023/050485

§ 371 Date: Sep. 6, 2024

(30) Foreign Application Priority Data
Mar. 8, 2022 (GB) 2203179.3

(51) Int. Cl. A61B 1/24; A46B 15/00; A61B 1/00; A61B 1/04; A61B 1/06

(52) U.S. Cl. CPC A61B 1/24; A46B 15/0004; A46B 15/0036; A61B 1/00006; A61B 1/000094; A61B 1/043; A61B 1/063; A61B 1/0684; A46B 2200/1066

(57) ABSTRACT

The present invention provides an interdental gap detection system for an oral inspection device. The gap detection system comprises a light emission module configured to emit light to irradiate an oral region of interest. The gap detection system comprises an optical filter arranged to preferentially filter, from light reflected from or emitted by the oral region of interest, fluorescence emitted from oral structures in the oral region of interest. The gap detection system comprises a sensor module configured to detect the filtered light and output corresponding sensor data. The gap detection system further comprises a processor module configured to process the sensor data output by the sensor module, to identify the presence of an interdental gap in the oral region of interest.

### FIELD OF THE INVENTION

The present invention relates generally to devices and methods for oral inspection, and in particular, for inspection of oral structures. In particular, but not exclusively, the present disclosure concerns gap detection systems for detecting gaps between oral structures (e.g. for detecting gaps between teeth), as well as to devices incorporating such systems, and uses of such systems.

### BACKGROUND

Various systems comprising a camera for oral inspection are known (sometimes referred to as ‘dental intra-oral camera systems’). These devices are generally used for imaging of the teeth, gums, or other oral structures of a patient in a medical setting—for example to identify one or more oral structures such as teeth, or to distinguishing between abnormal and healthy tooth surfaces by identifying plaques or caries on or within teeth, to determine whether one or more treatments should be applied to the oral structures.

However, there are a number of problems to be overcome with respect to use of conventional imaging systems for use in oral inspection. One primary issue is that in the environment of a user's mouth, and in particular during a dental cleaning process, multiple obscuration mediums may be present: for example, water, salvia, toothpaste and foam. Accordingly, when such obscurants are present, it may be more difficult to accurately identify, or distinguish between, different oral structures in a user's mouth.

Indeed, it may be particularly desirable to be able to detect an interdental gap between a user's teeth, for example for the purpose of observing a health state of the teeth adjacent the interdental gap location, or for applying a treatment to the interdental gap. In view of the above problem relating to visual identification of an interdental gap due to obscurants which may be present in an oral environment, interdental detection systems typically require physical probing of, or direct contact with, a user's teeth in order to identify an interdental gap.

For example, EP3888589A1 discloses an interdental space detection component for use with an interdental care device, which includes an interdental probe insertable into an oral cavity of a user for interdental space detection.

It would be desirable to provide a solution for identifying an interdental gap which does not require physical interaction with a user's teeth, but that is also able to satisfactorily identify the gap even in the presence of one or more obscuration mediums.

The present invention has been devised in light of the above considerations.

### SUMMARY OF THE INVENTION

The present inventors have realised that it may be possible to make use of the fluorescence properties of teeth, in order to provide an optical system for interdental gap detection which does not require physical interaction with a user's teeth, but that is also able to satisfactorily identify an interdental gap even in the presence of one or more obscuration mediums.

It is known that certain oral structures, and other materials which may be present in or around oral structures, may emit fluorescence upon irradiation with light of a specified wavelength. This has previously been used for different applications, to assist in distinguishing between abnormal and healthy tooth surfaces by identifying plaques or caries on or within teeth distinguishing between abnormal and healthy tooth surfaces during oral inspection. For example, KR 20210003373 A discloses an electric toothbrush having a toothbrush head portion which includes a UV LED module for irradiating teeth with ultraviolet rays, a fluorescent filter selectively transmitting the fluorescence emitted from the teeth by the ultraviolet rays from the UV LED module, and a camera module for imaging or storing the fluorescence filtered from the fluorescent filter. However, this document contains no disclosure on how an interdental gap can be detected with this system.

Accordingly, in a first aspect, the present invention provides an interdental gap detection system for an oral inspection device, the gap detection system comprising:
a light emission module configured to emit light to irradiate an oral region of interest;
an optical filter arranged to preferentially filter, from light reflected from or emitted by the oral region of interest, fluorescence emitted from oral structures in the oral region of interest; and
a sensor module configured to detect the filtered light and output corresponding sensor data;
wherein the system further comprises a processor module configured to process the sensor data output by the sensor module, to identify the presence of an interdental gap in the oral region of interest.

In a second aspect, the present invention provides an oral inspection and/or treatment device incorporating the gap detection system.

The oral inspection and/or treatment device may comprise a body and a head. In some embodiments, it may be a dental cleaning appliance comprising a body and a cleaning tool head. For example, it may be a toothbrush.

The present inventors have found that provision of such an arrangement can provide for improved detection of interdental gaps in comparison to known systems. In particular, the system does not require physical interaction with a user's teeth. Furthermore, it is also able to satisfactorily identify interdental gaps even in the presence of one or more obscuration mediums. In fact, it has been found that the presence of one of more obscuration mediums (e.g. foaming toothpaste) may even enhance the detection of interdental gaps in the present system, because such obscurants will typically flow into, or sit within the interdental gaps in a user's oral cavity. This may enhance the visual distinction between interdental gaps and other oral structure when visualised using a gap detected system according to the present invention.

We note that the term “interdental gap” is synonymous with the term “interproximal gap”. Accordingly, these terms are used interchangeably in the present disclosure.

The term “oral structures” is used to refer to physical structures present in an oral cavity, for example teeth. In preferred embodiments, the optical filter is arranged to preferentially filter fluorescence emitted from teeth in the oral region of interest.

An optical filter is a device that preferentially transmits light of at least some wavelengths, whilst reducing or prevent transmission of light of at least some other wavelengths. The present inventors have found that by arranging an optical filter which preferentially filters fluorescence emitted from oral structures such as teeth in the oral region of interest, identification of the presence of an interdental gap in the oral region of interest can be improved. The term “preferentially filters” is used herein to define that the filter is configured to preferentially absorb light within a set or range of predetermined wavelengths. Here, as the optical filter preferentially filters fluorescence emitted from oral structures such as teeth, the filter is configured to configured to preferentially absorb light in a wavelength range which includes one or more emission wavelengths of such fluorescence (for example, teeth typically emit fluorescence in a range having a peak at around 440 nm).

In some embodiments, the optical filter may be configured to preferentially absorb blue light. That is, it may be configured to preferentially absorb light having a wavelength in a range of from about 400 nm to about 495 nm, preferably around 440 nm to around 495 nm. This may be achieved by selecting an appropriate colour for the optical filter, in particular where the filter is a bulk-dyed filter. Accordingly, in some embodiments, the optical filter may be a yellow filter, or a yellow-green filter. A yellow or yellow-green filter will preferentially transmit light having wavelengths above around 500 nm, or above around 520 nm, and will preferentially filter (or ‘block’) blue light having shorter wavelengths than this. In other words, the optical filter may be configured to allow for preferential transmission of light having a wavelength of 500 nm or more, 510 nm or more, 520 nm or more, 530 nm or more, or 540 nm or more. The optical filter may have an upper bound for such preferential transmission, e.g. of around 600 nm. Provision of such a filter can provide a perceived normalisation of the reflected and fluoresced light, by provide a perceived reduction in blue reflected or emitted light of around 50%, thereby improving gap detection based on sensor data output by a sensor module configured to detect light reflected from or emitted by the oral region of interest. In some embodiments, at least 5%, at least 10%, at least 20%, at least 30%, at least 40%, at least 50%, at least 60%, at least 70%, at least 80% or at least 90% of fluoresced light emitted from selected oral structures in the oral region of interest may be removed by the filter. In embodiments, up to 100% of fluoresced light emitted from selected oral structures in the oral region of interest may be removed by the filter.

The specific form of the optical filter used in the present invention is not particularly limited, other than it must be suitable for being arranged in an optical path between the oral region of interest and the sensor module. In some embodiments, the optical filter may be a bulk-dyed filter. In other embodiments the optical filter may comprise an interference coating. In some embodiments the filter may comprise an absorptive filter, an interference filter, or a dichroic filter. Preferably the filter comprises an absorptive filter, as absorptive filters are generally relatively low cost and readily available. The optical filter may be made from any suitable material: for example it may be made from a glass material or a polymeric material (such as a resin).

As noted above, the light module is configured to emit light to irradiate an oral region of interest. The light emission module may be configured to emit light having a wavelength in a visible part of the electromagnetic spectrum, and/or light in a non-visible part of the electromagnetic spectrum.

The present inventors have realised that known arrangements for oral inspection which utilise fluorescence properties of oral structures typically use light emission modules which are configured to emit light having a wavelength lying in the UV region. That is, light having a wavelength up to around 400 nm, for example in a range of from about 380 nm to about 400 nm, with this range of wavelengths being selected to allow for suitable detection of plaque and/or caries legions. However, the present inventors have realised that selection of a different range of longer wavelengths for the emission light may allow for suitable detection of interdental gaps, with the possibility of reduced health risk to a user.

Accordingly, in some embodiments, the light emission module is configured to emit light in a ‘near-UV’ range. That is, the light emission module may be configured to emit light having a wavelength in a range of from 405 to 450 nm. Preferably, the light emission module does not emit light having a wavelength in a UV rage (i.e. at wavelengths of 400 nm or less). By providing an emission wavelength of 405 nm or more, the health risk to a user of the device may be reduced. Accordingly, the light emission module may be configured to emit light having a wavelength of 405 nm or more, 410 nm or more, 415 nm or more or 420 nm or more. However, it may be desirable for the emission wavelength not to be too high. This is because light of higher wavelengths may not have sufficient energy to excite fluorescence of teeth. Accordingly in some embodiments, the light emission module may be configured to emit light having a wavelength of 450 nm or less, 440 nm or less, or 430 nm or less. In some preferred embodiments, the light emission module may be configured to emit light having a wavelength in a range of from 405 to 420 nm.

The specific form of the light emission module is not particularly limited. In some embodiments, the light emission module may comprise one or more light emitting diodes, LEDs, for example. In other embodiments, the light emission module may comprise a laser light source. In some embodiments, the light emission module may be located near, or adjacent to, the sensor module.

The precise form of the sensor module is not particularly limited, provided that it is configured to detect light reflected from or emitted by the oral region of interest and output corresponding sensor data. In some embodiments, the sensor module comprises a camera or light detector. The sensor data output by the sensor module will typically comprise image data generated by the sensor module. Accordingly the phrases “output sensor data” and “generated image data” are used interchangeably in the following disclosure. In some embodiments, the generated image data (output sensor data) comprises red, green and blue, RGB, image data. The use of RGB image data may allow for more accurate gap localisation compared to other types of image data. Other types of image data (e.g. black and white image data) may be used in alternative embodiments.

As mentioned above, the gap detection system may be incorporated in an oral inspection device comprising a body and a head (e.g. it may be a dental cleaning appliance comprising a body and a cleaning tool head). In some embodiments, the light emission module and/or the sensor module are at least partially comprised in the head of the device. Alternatively or additionally, the light emission module and/or the sensor module may be at least partially comprised in the body of the device. By arranging the image sensor equipment at least partially in the body of the device, on-device space may be managed more efficiently. That is, the head of the device may be relatively small compared to the body. Further, in embodiments, the head of the device may be separable from the body. It may also be disposable, and it may be desired for a user to replace the head periodically after use. Arranging the light emission module and/or the sensor module at least partially in the body, as opposed to entirely in the head, may thus reduce the cost of replacement parts. Where the light emission module and/or the sensor module may be at least partially comprised in the body of the device, one or more guide components may be provided to conduct light from/to the light emission module and/or the sensor module. The guide components may comprise one or more fibre optic cables.

In some embodiments, the oral inspection device comprises a head, and the sensor module comprises; a sensor; and an aperture for receiving light and delivering the light to the sensor, wherein the aperture is comprised in the head of the oral inspection device. This allows the aperture to receive light within the oral cavity of the user, to detect light reflected from or emitted by the oral region of interest. As mentioned above, the sensor module may then comprise a guide channel for guiding light from the aperture to the sensor of the sensor module. For example, where the oral inspection device comprises a head, a body, and a stem connecting the head and the body, the guide channel may extend from the aperture to the sensor, along (e.g. within) the stem. The guide channel may comprise a fibre optic cable, for example. The sensor module may comprise a plurality of image sensors in some cases.

As mentioned above, the system further comprises a processor module configured to process the sensor data output by the sensor module, to identify the presence of an interdental gap in the oral region of interest. The processor may form part of a controller of the system. In other words, the system may comprise a controller which is operable to perform various data processing and/or control functions by means of one or more processors. The controller and/or processor forming part of the controller may additionally be operable to perform various other data processing and/or control functions in addition to processing of the sensor data output by the sensor module to identify the presence of an interdental gap in the oral region of interest. Operations performed by the processor module may be carried out by hardware and/or software.

The processor may be configured to identify the presence of an interdental gap in the oral region of interest based on (i) identification of a difference in the amount of light reflected or emitted from a first region as compared with the amount of light reflected or emitted from a second region, and/or (ii) based on a perceived or measured colour difference between a first region and a second region (e.g. as determined by comparison of colour values such as RGB colour values in the first region as compared with the second region).

This step of identification the presence of an interdental gap in the oral region of interest may constitute a step of determining location data indicating a location of an interproximal gap between adjacent teeth in the oral cavity of the user.

In some embodiments, the processor may be configured to process the output sensor data/generated image data using a trained classification algorithm configured to identify interproximal gaps, the trained classification algorithm having been trained prior to the use of the gap detection system.

Processing intraoral image data using the trained classification algorithm may increase the accuracy and/or reliability of interproximal gap detection and/or localisation compared to a case in which intraoral image data is not processed using such a trained algorithm. This may enable a finer and/or more intelligent control of the gap detection system, or oral inspection device incorporating the gap detection system. By more accurately detecting the interproximal gap and/or the position of the device relative to the interproximal gap during use of the device, it may be possible to more accurately perform one or more functions which rely on identification of the gap. For example, the oral inspection device incorporating the gap detection system may be configured to apply a treatment to the identified interdental gap in the oral region of interest, and accordingly, by more accurate identification of the identified interdental gap, treatment delivery may be made more accurate. This can improve the reliability and/or functionality of the device. Further, the use of image data allows the gap to not only be detected, but also localised. Localising the gap allows for a more accurate and/or reliable treatment delivery compared to a case in which the gap is not localised. In embodiments, the determined location of the gap comprises a location within the oral cavity of the user.

In embodiments, the processor is configured to process the generated image data using a sliding window. In such embodiments, the location data is determined by detecting the presence of the interproximal gap within the sliding window. This allows the interproximal gap to not only be detected, but localised, e.g. by determining a sub-region of the image which contains the gap.

In embodiments, the processor is configured to process the generated image data by extracting one or more image features from the image data and using the extracted one or more image features to determine the location data. This may further improve the accuracy of gap localisation. In embodiments, the processor is configured to extract the one or more image features using a discrete wavelet transform. Features extracted using a discrete wavelet transform may be used to more accurately detect and localise an interproximal gap from image data. In embodiments, the processor is configured to extract the one or more image features using at least one of: an edge detector, a corner detector, and a blob extractor. Extracting image features using such methods may provide a more accurate detection and/or localisation of interproximal gaps compared to other methods.

The processor module may be configured to output a signal on identification of the presence of an interdental gap in the oral region of interest. The precise nature of such signal is not particularly limited. In some embodiments, the gap detection system may be configured to perform one or more ancillary actions based on a signal output by the processor module. This is particularly relevant for embodiments where the gap detection system is provided as part of an oral inspection and/or treatment device. For example, an oral treatment device incorporating the gap detection system may be configured to apply a treatment to the identified interdental gap in the oral region of interest upon identification of the presence of an interdental gap in the oral region of interest. In some embodiments the oral inspection or treatment device may be a dental cleaning appliance, such as a toothbrush.

An oral treatment device incorporating the gap detection system may in some embodiments comprise a fluid delivery system for delivering working fluid to the oral cavity of the user. In such embodiments, a controller of the device (for example, the controller incorporating the processor module of the gap detection system) may be configured to output a control signal to the fluid delivery system to control delivery of the working fluid based on location data relating to an interdental gap identified by the processor module. As such, delivery of the working fluid during use of the device may be controlled in response to the automatic localisation of the interproximal gap (during the same use of the device) achieved using the sensor module and trained classification algorithm. This may allow for a more accurate and/or reliable use of the fluid delivery system. In particular, the accuracy of fluid jetting, i.e. the likelihood that working fluid is actually jetted into the interproximal gap, as opposed to elsewhere, may be increased. By improving the accuracy and/or reliability of the fluid delivery system, working fluid usage may be reduced and more effective treatment achieved more quickly.

In some embodiments the gap detection system, or the oral inspection device/oral treatment device incorporating the gap detection system, may further comprise a user interface. In some such embodiments, the processor module may be configured to cause the user interface to provide an output dependent on the location data derived from identification of the interdental gap in the oral region of interest. For example, the output may comprise a notification notifying the user that an interproximal gap has been located, indicating the location of the interproximal gap, informing the user that a treatment delivery has been performed on the interproximal gap, and/or instructing the user to adjust the position and/or orientation of the device such that more accurate treatment delivery (e.g. jetting of working fluid) can be performed.

In embodiments, the gap detection system, or the oral inspection device/oral treatment device incorporating the gap detection system comprises a memory. The memory may be configured for storage of one or more characteristics of the interproximal gap in the memory for use in subsequent processing and/or control of the gap detection system, or oral inspection/treatment device. For example, the one or more stored characteristics may be used to compare the interproximal gap with subsequently identified interproximal gaps. By distinguishing between different interproximal gaps of a user, repeated treatment of a given gap may be avoided, if so desired, thus reducing working fluid usage. In other cases, the one or more stored characteristics are used to track the interproximal gap over time.

The physical location of the processor in the gap detection system is not particularly limited. In some embodiments, the processor and/or a controller incorporating the processor may be provided locally. For example, where the gap detection system is incorporated in an oral inspection or treatment device, the processor and/or a controller may be located within the device, e.g. in a body of the device.

Alternatively, the processor and/or a controller incorporating the processor may be provided remotely, e.g. as part of a remote device. In such arrangements, gap detection system may further comprise means for communication with the remote device, to allow for transmission of the sensor data output by the sensor module to the processor module. The gap detection system may comprise means for communication with the remote device via a network, e.g. ‘the Cloud’. Providing the processor module in a remote device may allow for more efficient management of on-device space.

In a third aspect of the present disclosure, the present invention provides a method for detecting an interdental gap in an oral region of interest, including steps of:
irradiating an oral region of interest with light;
detecting light reflected from or emitted by the oral region of interest, said light having passed through an optical filter arranged to preferentially filter fluorescence emitted from oral structures in the oral region of interest;
outputting sensor data corresponding to the detected light; and
processing said sensor data to identify the presence of an interdental gap in the oral region of interest.

The step of irradiating an oral region of interest with light may be performed using a light emission module configured to emit light having a wavelength in a range of from 405 to 450 nm.

The step of detecting light reflected from or emitted by the oral region of interest may be performed using a sensor module configured to detect light reflected from or emitted by the oral region of interest. The same sensor module may perform the subsequent step of outputting sensor data corresponding to the detected light. The output sensor data may comprise image data representing at least a portion of the region of interest. As noted above, the detected light should have passed through an optical filter arranged to preferentially filter fluorescence emitted from oral structures in the oral region of interest. Accordingly, in some embodiments, the method also includes a step of arranging said optical filter.

The step of processing said sensor data to identify the presence of an interdental gap in the oral region of interest may be performed using a processor module. This step of identification the presence of an interdental gap in the oral region of interest may constitute a step of determining location data indicating a location of an interproximal gap between adjacent teeth in the oral cavity of the user.

The invention includes the combination of the aspects and preferred features described except where such a combination is clearly impermissible or expressly avoided. For example, a method of the invention may incorporate any of the features described with reference to an apparatus of the invention and vice versa.

### SUMMARY OF THE FIGURES

Embodiments and experiments illustrating the principles of the invention will now be discussed with reference to the accompanying figures in which:

FIG. 1. is a schematic drawing of a gap detection system according to the present invention.

FIG. 2. is a schematic drawing of an oral treatment device according to the present invention.

FIG. 3. is a flow diagram showing a method of operating an oral treatment device according to the present invention.

FIG. 4. shows illumination of an oral region of interest in a model of a human oral cavity (a) without, and (b) with an optical filter arranged to preferentially filter fluorescence emitted from oral structures in the oral region of interest.

FIG. 5. shows oral structures under various conditions of illumination, with and without the presence of obscurants.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

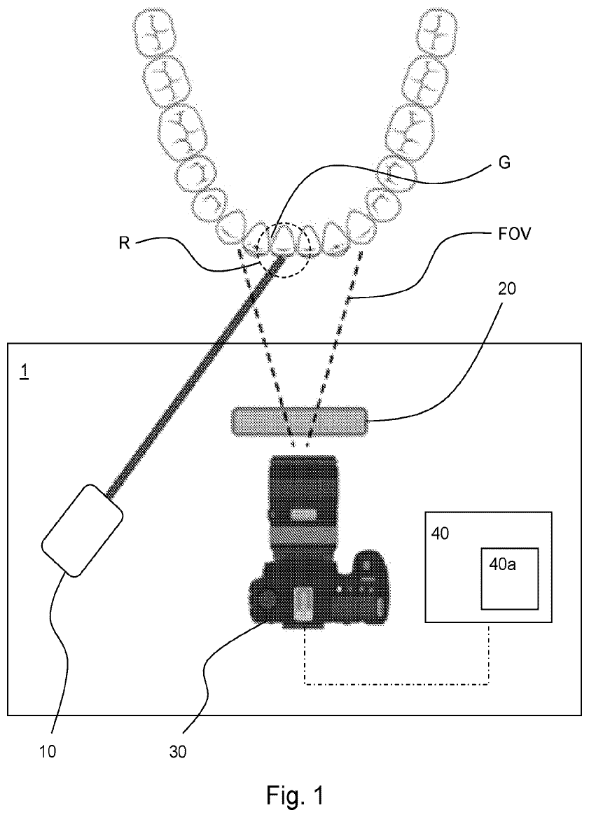

FIG. 1. is a schematic drawing of a gap detection system 1 according to the present invention, arranged to identify the presence of an interdental gap G within an oral region of interest R in the oral cavity of a user. In the schematic drawing shown, it can be seen that components of the system are not incorporated in a single device, but rather, are provided as a plurality of separate components. The system includes a light emission module 10, an optical filter 20, a sensor module 30, and a controller 40 including a processor module 40a.

The light emission module 10 is here conveniently provided as a laser which is configured to emit light in a ‘near-UV’ range, at a wavelength of 405 nm. The laser is arranged to irradiate an oral region of interest R within an oral cavity. Here the oral region of interest includes various oral structures including one or more teeth. Irradiating the oral region of interest with light having a wavelength of 405 nm allows for fluorescence of teeth which are present within the oral region of interest. However, because the light has a wavelength which lies in the ‘near-UV’ region of the electromagnetic spectrum, rather than a wavelength that lies in a UV range of the electromagnetic spectrum, the health risk to a user of the device may be reduced. In some preferred embodiments, the wavelength of light emitted by the light emission module is greater than 405 nm, for example 410 nm or more—this may further reduce any health risk to the user.

The optical filter 20 in this embodiment is a bulk-dyed polymeric filter having a yellow colour. The optical filter is arranged to lie in an optical path between the oral region of interest and the sensor module (as indicated by the dashed lines showing the field of view (FOV) of the sensor module).

This yellow filter will preferentially transmit light having wavelengths above around 500 nm and will preferentially filter (or ‘block’) light having shorter wavelengths than this. This means that the filter will preferentially absorb light in a wavelength range which includes one or more emission wavelengths of fluorescence emitted by oral structures such as teeth, which typically emit fluorescence in a range having a peak at around 440 nm. Provision of such a filter can provide a perceived normalisation of the reflected and fluoresced light, by provide a perceived reduction in blue reflected or emitted light of around 50%, thereby improving gap detection based on sensor data output by a sensor module configured to detect light reflected from or emitted by the oral region of interest.

The sensor module 30 is represented in this schematic figure by a ‘camera’ icon. In practice, the form of the sensor module is not particularly limited, however it preferably comprises one or more image sensors. Examples of such image sensors include, but are not limited to, charge-coupled devices, CCDs, and active-pixel sensors such as complementary metal-oxide-semiconductor, CMOS, sensors. Whilst in this embodiment the sensor module is a standalone component arranged to be used outside the oral cavity, in some embodiments, the sensor module is an intraoral sensor—that is, a sensor module operable to be used at least partially inside the oral cavity of a user, in order to generate image data representing the oral cavity of the user. For example, as will be discussed in further detail below, when the gap detection system is incorporated in an oral treatment device the sensor module may be at least partially arranged on a head of the oral treatment device which is arranged to be inserted into the oral cavity of the user.

In the present embodiment, as the sensor module 30 is provided as a standalone component, the sensor module comprises one or more processors (not shown). The sensor module is configured to detect light reflected from or emitted by the oral region of interest R within the field of view FOV of the sensor module, said light having passed through the optical filter 20. The sensor module is configured to output sensor data corresponding to the detected light, which in this case will be generated image data. The generated image data (output sensor data) in this embodiment comprises red, green and blue, RGB, image data. The use of RGB image data may allow for more accurate gap localisation compared to other types of image data.

The sensor module is configured to transmit the output sensor data to the controller 40, as indicated by the dashed line connecting these two components. This transmission may be performed via wires physically connecting the sensor module 30 and the controller 40 or may be by wireless transmission. The controller 40 is operable to perform various data processing and/or control functions, as will be described in more detail below. The controller 40 may comprise one or more components. The one or more components may be implemented in hardware and/or software. The one or more components may be co-located or may be located remotely from each other. The controller 40 may be embodied as one or more software functions and/or hardware modules. The controller 40 comprises at least one processor 40a configured to process instructions and/or data—and in particular, configured to process sensor data which is output from the sensor module 30. Operations performed by the one or more processors 40a may be carried out by hardware and/or software. The controller 40 and/or processor module 40a of the controller are operable to output control signals for controlling one or more components of the gap detection system 1, or for controlling an oral treatment device incorporating the gap detection system.

The processor 40a may be part of a processing system comprising one or more processors and/or memory. The one or more processors of processing systems may comprise a central processing unit (CPU). The one or more processors may comprise a graphics processing unit (GPU). The one or more processors may comprise one or more of a field programmable gate array (FPGA), a programmable logic device (PLD), or a complex programmable logic device (CPLD). The one or more processors may comprise an application specific integrated circuit (ASIC). It will be appreciated by the skilled person that many other types of device, in addition to the examples provided, may be used to provide the one or more processors. The one or more processors may comprise multiple co-located processors or multiple disparately located processors. Operations performed by the one or more processors may be carried out by one or more of hardware, firmware, and software. It will be appreciated that processing systems may comprise more, fewer and/or different components from those described.

In embodiments, the processor 40a processes the sensor data output by the sensor module by means of one or more data analysis algorithm(s). The data analysis algorithm(s) may be configured to analyse received data, e.g. image data, and produce an output useable as a condition by which the gap detection system 1, or an oral treatment device incorporating the gap detection system, may be controlled. For example they may be configured to output a signal indicative of the presence of a gap or no gap in the oral region of interest. In embodiments, the data analysis algorithm(s) comprises a classification algorithm, e.g. a non-linear classification algorithm. In embodiments, the data analysis algorithm(s) comprises a trained classification algorithm. However, in alternative embodiments, the data analysis algorithm(s) comprises other types of algorithm, e.g. not necessarily trained and/or not configured to perform classification.

In embodiments, the processor 40a is configured to process the generated image data using a sliding window, which allows for location data of an interdental gap to be determined by detecting the presence of the interdental gap within the sliding window, and determining a sub-region of the image which contains the gap. In a sliding window image analysis technique, the sliding window passes across the image, defining sub-regions of the image, and a determination is made on whether a gap exists in each sub-region of the image. This is described in more detail below.

In embodiments, the generated image data is processed by extracting one or more image features from the image data, and using the extracted one or more image features to determine the location data. The image features may comprise texture-based image features, for example.

Since an image consists of pixels which are highly related to each other, image feature extraction is used to obtain the most representative and informative (i.e. non-redundant) information of an image, in order to reduce dimensionality and/or facilitate learning of the classification algorithm.

In embodiments, the one or more image features are extracted using a discrete wavelet transform. The discrete wavelet transform can capture both frequency and location information in an image. The image frequency in gap areas is typically higher than the image frequency in teeth or gum areas. This allows a discrete wavelet transform to produce a frequency map of the image that is usable to detect interproximal gaps. In embodiments, a Haar wavelet is used, which has a relatively low computational complexity and low memory usage compared to other wavelets. The coefficients of the wavelet transform (or approximations thereof) may be used as the extracted image features. For example, the output of feature extraction based on the Haar wavelet applied to an image of size a×a may include a horizontal wave h (a/4×a/4), a vertical wave v (a/4×a/4) and a diagonal wave d (a/4×a/4). Other wavelets can be used in alternative embodiments.

The extracted features may be used for a sliding window applied to the image. For example, in the image sub-region defined by the sliding window, a 2×2 pooling for each of h, v and d may be performed, before h, v and d are vectorised and combined into one vector with size 1×108. This may be normalised, along with trained data from the trained classification algorithm, e.g. trained mean and variance values. A support-vector machine, SVM, may be used as a non-probabilistic non-linear binary classifier with a Gaussian radial basis function kernel, which receives the normalised data from the previous step. The trained SVM comprises support vectors having trained coefficients and biases, i.e. determined during a previous training phase. For example, given a set of images together with ground truth labelling, the classification algorithm can be trained so as to assign new examples to one category (e.g. gap) or another (e.g. non-gap).

In embodiments, the one or more image features are extracted using at least one of: an edge detector, a corner detector, and a blob extractor. Extracting image features using such methods may provide a more accurate detection and/or localisation of interproximal gaps compared to other methods.

In embodiments, the generated image data is processed using a trained classification algorithm configured to detect interproximal gaps. Using such a trained algorithm results in a more accurate and/or reliable gap detection compared to a case in which a trained algorithm is not used. In embodiments, the classification algorithm comprises a machine learning algorithm. Such a machine learning algorithm may improve (e.g. increase accuracy and/or reliability of classification) through experience and/or training.

In embodiments, the generated image data is processed to determine at least one characteristic of the identified interproximal gap. In embodiments, the at least one characteristic is determined by processing the generated image data using a machine learning algorithm. The machine learning algorithm is trained to identify information for use in distinguishing between interproximal gaps. Such information comprises features that are representative of the gap, i.e. non-redundant features, and/or features which are predicted to vary between gaps. The identified information may comprise the at least one characteristic of the gap. In embodiments, such a machine learning algorithm (or one or more different machine learning algorithms) is also used to determine the at least one characteristic of the one or more previously identified gaps, e.g. features that are representative of the previously identified gaps and/or useable to distinguish between gaps, and which are used to compare the previously identified gaps with the currently identified gap. In alternative embodiments, characteristic features of the gaps are extracted from raw image data without the use of machine learning algorithms.

In embodiments, the classification algorithm is modified using the output signals and/or the generated image data. That is, the classification algorithm may be trained and/or further trained using the generated signals and/or the generated image data. Modifying the classification algorithm allows the accuracy and/or reliability of the algorithm to improve through experience and/or using more training data. That is, a confidence level of the determined interdental gap location may be increased. Further, modifying the classification algorithm allows the classification algorithm to be tailored to the user. By using the generated signals and/or the generated image data as training data to dynamically re-train the classification algorithm, the classification algorithm can more reliably determine the intraoral location of an interdental gap.

In embodiments, training data is received from a remote device. The training data may be received from a network, e.g. ‘the Cloud’. Such training data may comprise classification data associated with other users. Such training data may comprise crowd-sourced data, for example. In embodiments, such training data may be greater in volume than classification data obtained using the gap detection system directly. The use of the training data from the remote device to modify the classification algorithm can increase the accuracy and/or reliability of the classification algorithm compared to a case in which such training data is not used.

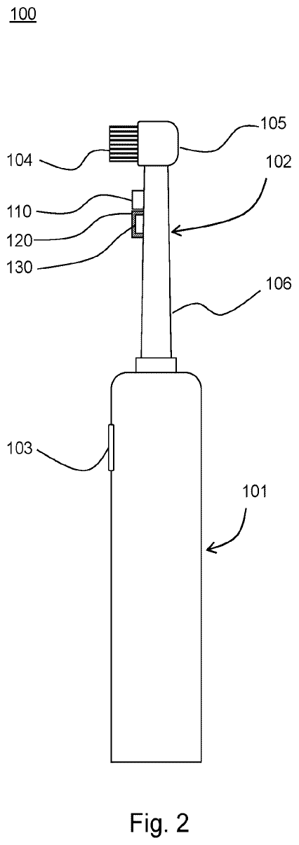

Gap detection systems according to the present invention may be implemented in oral treatment devices. FIG. 2 is a schematic drawing of an oral treatment device according to the present invention. The oral treatment device in the present instance is a toothbrush, although in other embodiments the oral treatment device may be a flossing device, an oral irrigator, an interproximal cleaning device, an oral care monitoring device, or any combination of such.

In this embodiment, the oral treatment device 100 comprises a handle 101 and a tool head 102. The handle 101 forms the main body of the device 100 and may be gripped by a user during use of the device 100. It is generally cylindrical in shape. In the embodiment shown in FIG. 2, the handle 101 comprises a user interface 103. The user interface 103 comprises a user operable button configured to be depressible by the user when the user is holding the handle. This may be operable to control e.g. a power state of the oral treatment device 100, or to provide user input to the oral treatment device in a known manner.

The tool head 102 comprises a plurality of bristles 104 for performing a tooth brushing function, although as discussed above, an oral treatment device according to the present invention may take other forms not requiring the presence of bristles. For example, in some other embodiments the oral treatment device 100 comprises a dedicated fluid delivery device, e.g. for performing a treatment such as cleaning gaps between adjacent teeth, and/or for delivering a cleaning or whitening medium to the teeth of the user. The tool head 102 comprises a head portion 105 and a stem portion 106. The stem portion connects the handle to the head portion of the tool head 102. The stem portion 106 is elongate in shape, which serves to space the head portion of the tool head from the handle to facilitate user operability of the oral treatment device 100. The head 102 and/or the stem 106 may be detachable from the handle 101.

As mentioned above, in this embodiment, the gap detection system is incorporated in the oral treatment device. Many components of the gap detection system are not shown in this figure.

However, a light emission module 110 in the form of an LED is provided on the stem portion 106 of the tool head 102. A sensor module 130 is also provided on the stem portion 106 of the tool head and is covered by an optical filter 120, so that the optical filter is arranged in an optical path between the oral region of interest and the sensor module. The advantage of providing the light emission module and/or the sensor module on the tool head 102 is that it allows for intraoral imaging in a facile manner.

Whilst in this embodiment the light emission module 110 and the sensor module 130 are provided on the tool head 102, it is also contemplated than in some embodiments, these components may be located in the handle/body 101 of the device, and one or more guide components may be used for receiving and delivering light to and from the light emission module 110 and the sensor module 130. For example, the device may comprise one or more guide channels extending from an aperture provided on the tool head 102, along (e.g. within) the stem portion 106, to the light emission module 110 and the sensor module 130 located in the handle of the device. In such an arrangement the guide channel may comprise one or more fibre optic cables.

It will be understood that the oral treatment device 100 may further comprise additional components that are not shown or described here, e.g. a power source such as a battery, or other components conventionally present in oral treatment devices such as electric toothbrushes, flossing devices, oral irrigators, interproximal cleaning devices, or oral care monitoring devices.

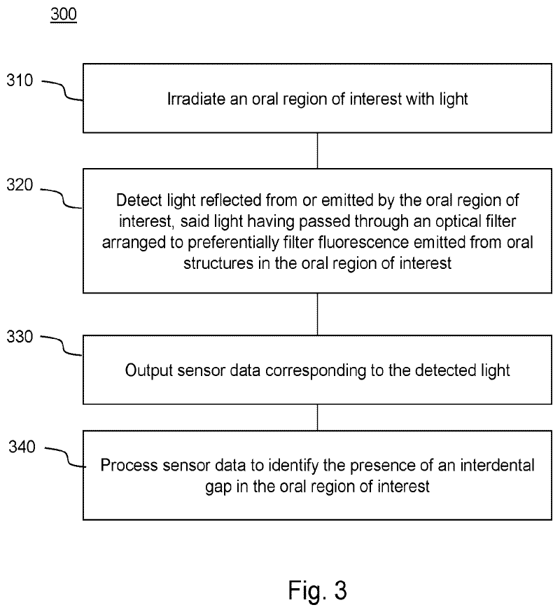

FIG. 3. is a flow diagram showing a method 300 of operating a gap detection system, or oral treatment device incorporating a gap detection system according to the present invention. The method 300 may be used to operate the gap detection system 1 or oral treatment device 100 described above with reference to FIG. 1 and FIG. 2. The method 300 is performed at least in part by the processor module 40a of the gap detection system.

In step 310, a light emission module 10, 110 is used to irradiate an oral region of interest with light. In line with the discussion of FIG. 1, in this embodiment the light is laser light having a wavelength of 405 nm, although other wavelengths may be used.

In step 320, the sensor module 30, 130 detects light reflected from or emitted by the oral region of interest, said light having passed through an optical filter 20, 120 arranged to preferentially filter fluorescence emitted from oral structures in the oral region of interest.

In step 330, the sensor module 30, 130 outputs sensor data (in this embodiment, generated image data representing at least a portion of the oral cavity of the user). The image data comprises red, green and blue (RGB) image data.

In step 340, the generated image data is processed to identify the presence of an interdental gap in the oral region of interest. In preferred embodiments, the data is processed to determine location data indicating a location of an interproximal gap between adjacent teeth in the oral cavity of the user. The generated image data is processed using a trained classification algorithm configured to identify interproximal gaps. The trained classification algorithm is trained prior to the use of the oral treatment device.

When the method is used for control of an oral treatment device, the method may include a further optional step (not shown) of controlling the oral treatment device 100 to deliver a treatment to the detected interdental gap.

By use of this method, an interdental gap can be detected automatically, through use of the sensor module and trained classification algorithm, and in a more accurate manner than may be possible using existing systems. Furthermore, in systems or devices configured for delivery of one or more treatments within an oral cavity (for example, in systems configured for jetting of fluid at or adjacent to identified oral structures), treatment delivery can be controlled accordingly, without the need for user input. This can have significant advantages. For example, the user does not need to determine when an oral treatment device incorporating the gap detection system is in a position that is suitable for treatment delivery, e.g. proximate to or in an interproximal gap. Instead, such a determination can be made automatically based on intraoral image data generated by the sensor module and can be performed in substantially real-time.

The method may be implemented at least in part by computer software stored in (non-transitory) memory and executable by the processor, or by hardware, or by a combination of tangibly stored software and hardware (and tangibly stored firmware). Embodiments also extend to computer programs, particularly computer programs on or in a carrier, adapted for putting the above-described embodiments into practice. The program may be in the form of non-transitory source code, object code, or in any other non-transitory form suitable for use in the implementation of processes according to embodiments. The carrier may be any entity or device capable of carrying the program, such as a RAM, a ROM, or an optical memory device, etc.

### Examples

In order to demonstrate application of the present invention, various experimental tests were conducted using a gap detection system as described above in relation to FIG. 1.

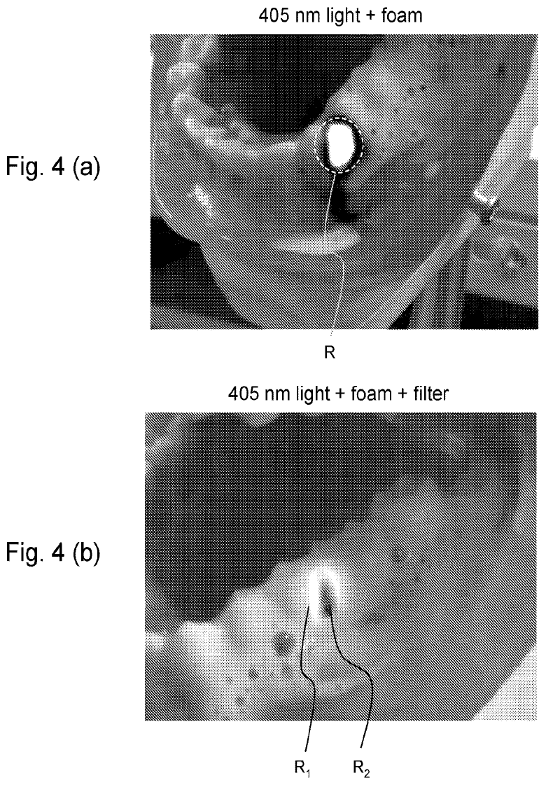

FIG. 4. shows illumination of an oral region of interest R in a model of a human oral cavity (a) without, and (b) with an optical filter arranged to preferentially filter fluorescence emitted from oral structures in the oral region of interest. Here, the oral region of interest was illuminated using a 405 nm (‘near-UV’) laser. It can be seen from FIG. 4 (a) that when no dedicated optical filter is present, the oral region of interest is brightly illuminated, and it is not possible to clearly distinguish between oral structures in the oral region of interest due to the significant amount of reflected and emitted light resulting from fluorescent of the teeth under illumination of the 405 nm laser.

In comparison, to obtain FIG. 4 (b), a yellow filter was arranged between the oral region of interest and the imaging sensor (here, the imaging sensor of an iPad Pro® camera having a sapphire crystal lens cover and hybrid IR filter). Provision of this yellow filter allows for a normalisation of the reflected light and fluoresced light, thereby improving the visual contrast between oral structures. Specifically, it provides a perceived reduction in blue reflected or emitted light of around 50%. A location of the interdental gap can be determined based on either the measured colour difference between a first region R₁ and a second region R₂ (region R₁ appearing to have a green colour, with RGB colour values similar to rgb (130, 174,65), region R₂ appearing to have a purple colour with RGB colour values similar to rgb (136,63,253)). Alternatively, a location of the interdental gap can be determined based on identification of a difference in the amount of light reflected or emitted from the first region as compared with the amount of light reflected or emitted from the second region, having passed through the optical filter (here, the amount of reflected/emitted light passing through the optical filter to reach the imaging sensor appears to be greater in the second region R₂). It can be seen that it is possible to determine the location of the interdental gap by identification of region R₂, even though an obscurant (a foaming toothbrush) is present in the model of the oral cavity.

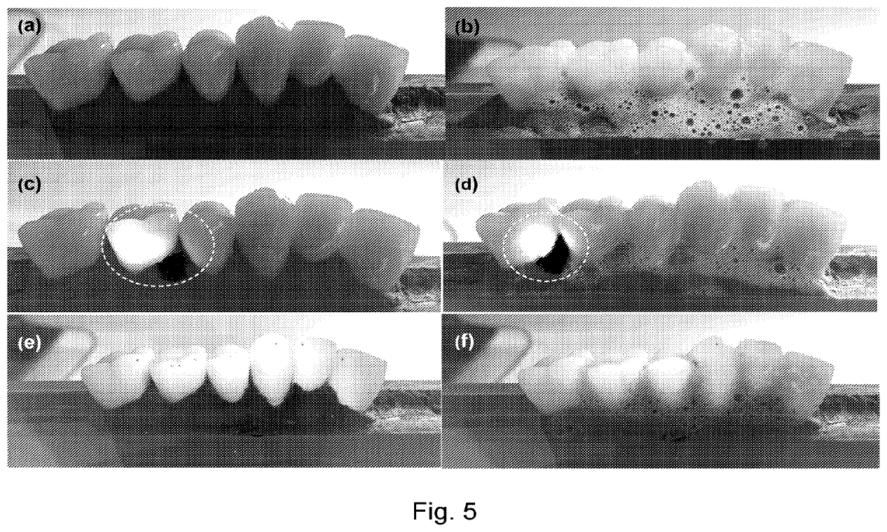

FIG. 5 shows oral structures under various conditions of illumination, with and without the presence of obscurants. Specifically, FIG. 5 (a) shows the oral structures without dedicated illumination, without an optical filter, and with no obscurants present; (b) without dedicated illumination, without an optical filter, and with obscurants (here, foam) present; (c) with laser illumination of the region indicated by the dashed circle by a 405 nm laser, with an optical filter, and with no obscurants present; (d) with laser illumination of the region indicated by the dashed circle by a 405 nm laser, with an optical filter, and with obscurants present; (e) with LED illumination of all oral structures by a 405 nm LED, with an optical filter, and with no obscurants present; and (f) with LED illumination of all oral structures by a 405 nm LED, with an optical filter, and with obscurants present.

Similarly to FIG. 4, from FIG. 5 it can be seen that provision of an optical filter can provide for high contrast distinction between oral structure in the oral cavity-specifically a high contrast distinction between teeth and interdental gaps. This was found to be the case independent of the specific mode of illumination (laser vs LED light). It can also be seen from a comparison of e.g. FIG. 5 (c) and FIG. 5 (d) that that the presence of an obscuration medium such as foaming toothpaste can enhance the visual contrast between oral structures, which can thereby allow for improved detection of interdental gaps.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

The techniques described herein may be implemented in software or hardware, or may be implemented using a combination of software and hardware. They may include configuring an apparatus to carry out and/or support any or all of techniques described herein. Although at least some aspects of the examples described herein with reference to the drawings comprise computer processes performed in processing systems or processors, examples described herein also extend to computer programs, for example computer programs on or in a carrier, adapted for putting the examples into practice. The carrier may be any entity or device capable of carrying the program. The carrier may comprise a computer readable storage media. Examples of tangible computer-readable storage media include, but are not limited to, an optical medium (e.g., CD-ROM, DVD-ROM or Blu-ray), flash memory card, floppy or hard disk or any other medium capable of storing computer-readable instructions such as firmware or microcode in at least one ROM or RAM or Programmable ROM (PROM) chips.

1. An interdental gap detection system for an oral inspection device, the gap detection system comprising:
a light emission module configured to emit light to irradiate an oral region of interest;
an optical filter arranged to preferentially filter, from light reflected from or emitted by the oral region of interest, fluorescence emitted from oral structures in the oral region of interest; and
a sensor module configured to detect the filtered light and output corresponding sensor data;
wherein the system further comprises a processor module configured to process the sensor data output by the sensor module, to identify the presence of an interdental gap in the oral region of interest.

2. The interdental gap detection system according to claim 1, wherein the light emission module is configured to emit light having a wavelength in a range of from 405 to 450 nm.

3. The interdental gap detection system according to claim 1, wherein the light emission module comprises an LED light source, or a laser light source.

4. The interdental gap detection system according to claim 1, wherein the optical filter is selected from a yellow filter, or a yellow-green filter.

5. The interdental gap detection system according to claim 1, wherein the filter is configured to preferentially absorb blue light, the blue light has a wavelength in a range of from about 400 nm to about 495 nm.

6. The interdental gap detection system according to claim 1, wherein the processor module is configured to identify the presence of an interdental gap in the oral region of interest, based on (i) identification of a difference in an amount of filtered light reflected or emitted from a first region as compared with the amount of filtered light reflected or emitted from a second region, or (ii) based on a perceived or measured colour difference between a first region and a second region.

7. The interdental gap detection system according to claim 1, wherein the processor module is configured to output a signal on identification of the presence of an interdental gap in the oral region of interest.

8. An oral inspection or treatment device incorporating the gap detection system according to claim 1.

9. The oral inspection or treatment device according to claim 8, wherein the oral inspection or treatment device is a dental cleaning appliance, further comprising a body and a cleaning tool head.

10. The oral inspection or treatment device according to claim 9, wherein one or both of the light emission module and the sensor module of the interdental gap detection system are provided on the cleaning tool head.

11. The oral inspection or treatment device according to claim 9, wherein the processor module is provided as part of a controller module located within the body of the dental cleaning appliance.

12. The oral inspection or treatment device according to claim 7, wherein the processor module is provided as part of a controller module located within a remote device, wherein the remote device and the oral inspection or treatment device are configured for communication with one another to allow for exchange of signals and/or data.

13. The oral inspection or treatment device according to claim 9 wherein, on identification of the presence of an interdental gap in the oral region of interest, the dental cleaning appliance is configured to apply a treatment to the identified interdental gap in the oral region of interest.

14. A method for detecting an interdental gap in an oral region of interest, including steps of:
irradiating an oral region of interest with light;
detecting light reflected from or emitted by the oral region of interest, said light having passed through an optical filter arranged to preferentially filter fluorescence emitted from oral structures in the oral region of interest;
outputting sensor data corresponding to the detected light; and
processing said sensor data to identify the presence of an interdental gap in the oral region of interest.

15. The method according to claim 14, wherein the light irradiating the oral region of interest has a wavelength in a range of from 405 to 450 nm.

16. The method according to claim 14, wherein the method is performed by an interdental gap detection system comprising:
a light emission module configured to emit light to irradiate an oral region of interest;
an optical filter arranged to preferentially filter, from light reflected from or emitted by the oral region of interest, fluorescence emitted from oral structures in the oral region of interest; and
a sensor module configured to detect the filtered light and output corresponding sensor data;
wherein the system further comprises a processor module configured to process the sensor data output by the sensor module, to identify the presence of an interdental gap in the oral region of interest.
